SEQUENCE LISTING (4 sequences)

SEQ ID NO: 1 - DNA, 20 bp, Artificial Sequence
  Description of Artificial Sequence Synthetic primer
accaccacacccagctaaat

SEQ ID NO: 2 - DNA, 20 bp, Artificial Sequence
  Description of Artificial Sequence Synthetic primer
cacgccctgctcatttttat

SEQ ID NO: 3 - DNA, 20 bp, Artificial Sequence
  Description of Artificial Sequence Synthetic primer
ccaagatccggaaagatgaa

SEQ ID NO: 4 - DNA, 20 bp, Artificial Sequence
  Description of Artificial Sequence Synthetic primer
gcatcttctaggtgccaggt

(19) United States
(12) Patent Application Publication
(10) Pub. No.: US 2014/0030219 A1
(43) Pub. Date: Jan. 30, 2014

(54) METHODS OF DIAGNOSING AND TREATING AGE-RELATED MACULAR DEGENERATION

(71) Applicants: Lai Wei, Rockville, MD (US); Robert Nussenblatt, Bethesda, MD (US); Baoying Liu, North Potomac, MD (US); Chi-chao Chan, Kensington, MD (US)

(72) Inventors: Lai Wei, Rockville, MD (US); Robert Nussenblatt, Bethesda, MD (US); Baoying Liu, North Potomac, MD (US); Chi-chao Chan, Kensington, MD (US)

(21) Appl. No.: 13/981,368

(22) PCT Filed: Jan. 25, 2012
(86) PCT No.: PCT/US12/22511

§ 371 Date: Oct. 8, 2013

Related U.S. Application Data
(60) Provisional application No. 61/435,989, filed on Jan. 25, 2011.

(51) Int. Cl. C12Q 1/68; G01N 33/68

(52) U.S. Cl. CPC C12Q 1/6883; G01N 33/6893
USPC 424/85.2; 435/6.11; 435/7.21; 506/9

(57) ABSTRACT

The present invention is directed to methods of diagnosing the presence, severity, or predisposition for development of age-related macular degeneration in a patient based on the frequency of IL-17RC+ cells and or methylation of the promoter of IL17RC gene. Methods of treated age-related macular degeneration are also described.

### CROSS REFERENCE TO RELATED APPLICATIONS

This application claims the benefit of U.S. Provisional Application No. 61/435,989, filed Jan. 25, 2011, the entirety of which is incorporated by reference herein.

### TECHNICAL FIELD

The present invention is directed to methods of diagnosing the presence, severity, or predisposition of age-related macular degeneration in a patient. Methods of treating age-related macular degeneration with soluble IL-17RC molecules are also described.

### BACKGROUND

Age-related macular degeneration (AMD) is the most common cause of irreversible blindness in the elder population worldwide. It currently affects more than 1.75 million individuals in the United States alone and this number will increase to almost 3 million in 10 years. AMD leads to progressive loss of central vision because of damage to the retina.

In light of the irreversible nature of the disease, methods of diagnosing AMD, as well as methods of identifying whether a patient is at risk of developing AMD are needed so medical intervention can be initiated as early into the disease as possible.

Currently, anti-Vascular Endothelial Growth Factor (anti-VEGF) drugs are the only available drugs for the treatment of neovascular wet AMD. Campa C, Harding S P. Anti-VEGF Compounds in the Treatment of Neovascular Age Related Macular Degeneration. Curr Drug Targets 2010; 2010:1. As such, alternative treatments are needed.

### SUMMARY

The present invention is directed to methods of diagnosing the presence, severity, or predisposition for development of age-related macular degeneration in a patient comprising determining the methylation of the promoter of the IL17RC gene in the DNA of patient having, suspected of having, or suspected to be at risk for age-related macular degeneration; and determining whether the promoter of the IL17RC gene is less methylated relative to the methylation of the promoter of the IL17RC gene in a control from an individual or individuals not having age-related macular degeneration, the less methylation being indicative of the presence, severity, or predisposition for development of age-related macular degeneration in the patient.

The invention is also directed to methods of diagnosing the presence, severity, or predisposition for development of age-related macular degeneration in a patient comprising determining the frequency of IL-17RC⁺ cells in a whole blood sample from a patient having, suspected of having, or suspected to be at risk for age-related macular degeneration and determining whether the blood sample has an elevation in the frequency of IL-17RC⁺ cells relative to the frequency of IL-17RC⁺ cells in a control from an individual or individuals not having age-related macular degeneration, the elevation being indicative of the presence, severity, or predisposition for development of age-related macular degeneration in the patient.

The invention is also directed to methods of treating age-related macular degeneration in a patient comprising administering to the patient a therapeutically effective amount of a soluble IL-17RC molecule.

### BRIEF DESCRIPTION OF THE DRAWINGS

FIG. 1A depicts MeDIP-chip data showing hypo-methylated CpG sites were found in AMD twins but not in non-AMD twins around the promoters of CCL22 and IL17RC genes.

FIG. 1B depicts methylation status on the promoters of CCL22 and IL17RC genes in siblings with discordant AMD.

FIG. 2 depicts that a hypo-methylated promoter of the IL17RC gene was found only in AMD patients, as compared to a hyper-methylated promoter of the IL17RC gene in healthy controls.

FIG. 3A depicts that increased levels of IL-17RC protein expression are found on several types of cells from AMD patients.

FIG. 3B depicts that increased levels of IL-17RC protein expression are found on CD14⁺ monocyte in an AMD patient.

FIG. 3C depicts that increased levels of IL-17RC expression are found on CD14⁺ monocyte in 28 AMD patients compared to 18 non-AMD controls.

FIG. 4A depicts that elevated expression of CXCR1, CXCR2, and CXCR4 mRNA in sorted IL17RC⁺CD14⁺ monocytes as compared to IL17RC-CD14⁺ monocytes.

FIG. 4B depicts that IL-17RC mRNA is detectable in the retinal and choroidal tissues from AMD patients but not of that from non-AMD controls.

### DETAILED DESCRIPTION OF ILLUSTRATIVE EMBODIMENTS

The present invention is directed to methods of diagnosing the presence, severity, or predisposition for development of age-related macular degeneration in a patient. According to the invention, these methods include determining the methylation of the promoter of the IL17RC gene in the DNA from a patient having, suspected of having, or suspected to be at risk for age-related macular degeneration; and determining whether the promoter of the IL17RC gene is less methylated relative to the methylation of the promoter of the IL17RC gene in a control from an individual or individuals not having age-related macular degeneration, the less methylation being indicative of the presence, severity, or predisposition for development of age-related macular degeneration in the patient.

In another embodiment, the diagnosis of the presence, severity, or predisposition for development of age-related macular degeneration in a patient is accomplished using methods that include determining the frequency of IL-17RC⁺ cells in a whole blood sample from a patient having, suspected of having, or suspected to be at risk for age-related macular degeneration; and determining whether the blood sample has an elevation in the frequency of IL-17RC⁺ cells relative to the frequency of IL-17RC⁺ cells in a control from an individual or individuals not having age-related macular degeneration, the elevation being indicative of the presence, severity, or predisposition for development of age-related macular degeneration in the patient.

Nomenclature used herein is consistent with that reported in Wain, H. M. et al. Guidelines for Human Gene Nomenclature Genomics 79(4):464-470 (2002.

As used herein, “IL17RC” refers to the IL17RC gene (Gene ID 84818), either DNA or RNA, or the locus of the gene.

As used herein, “IL-17RC” refers to the protein translated from the IL17RC gene.

Difference in DNA Methylation Patterns Between Twins with Discordant AMD

Three pairs of twins with phenotypic discordance of AMD were identified. Their age and gender information is listed in Table 1.

TABLE 1
Age and Gender of 212 Patients with AMD and 106 Controls without AMD

| | | | | |
|---|---|---|---|---|
| | | CFH | ARMS2 | HTRA1 |
| Gender | Ave | rs1061170 | rs10490924 | rs11200638 |
| (MF) | Age | (TT/TC/CC/unknown)* | (GG/GT/TT/unknown) | (GG/GA/AA/unknown) |

Twins

| | | | | | |
|---|---|---|---|---|---|
| Non-AMD1** | F | 88 | TC | TT | AA |
| AMD1 | F | 88 | TC | TT | AA |
| Non-AMD2 | F | 78 | CC | GG | GG |
| AMD2 | M | 78 | CC | GT | GA |
| Non-AMD3 | F | 79 | CC | GT | GA |
| AMD3 | F | 79 | CC | GT | GA |

Siblings

| | | | | | |
|---|---|---|---|---|---|
| Non-AMD | 4/3 | 65.9 | 0/6/1/0 | 2/2/2/1 | 1/2/0/4 |
| AMD | 2/5 | 63.4 | 0/4/3/0 | 0/3/3/1 | 0/1/0/6 |

Case-Control Patient Cohort

| | | | | | |
|---|---|---|---|---|---|
| Non-AMD | 45/51 | 70.2 | 41/39/16/0 | 61/30/5/0 | 59/29/8/0 |
| AMD | 91/111 | 80.5 | 40/93/67/2 | 57/90/44/11 | 58/89/49/8 |

To investigate the DNA methylation difference between the twins with discordant AMD, genomic DNA was obtained from their peripheral blood mononuclear cells (PBMCs) and subjected to MeDIP-chip analysis using NimbleGen Human DNA Methylation 2.1M Deluxe Promoter Array (Roche-NimbleGen, Madison Wis.), which includes probes tiling through 7250 bp upstream and 3250 bp downstream of transcription start sites of all genes and microRNAs. A total of 231 genes showed consistent differential methylation patterns on their promoters between AMD twins and their non-AMD co-twins. Consistent with the speculation that AMD might be an immunological disease, our gene ontology enrichment analysis using Ingenuity Systems (Redwood City, Calif.; http://www.ingenuity.com/) identified “Immunological Disease” as one of the 5 most significantly enriched gene categories among the list of genes with differential DNA methylation patterns between twins (Table 2).

TABLE 2
Enrichment of Disease Categories Among Genes with Differential
Promoter Methylation Patterns Between AMD and non-AMD Twins

| | | # of Molecules |
|---|---|---|
| Diseases and Disorders Name | Corrected P value | in list |
| Immunological Disease | 4.13E−02 | 58 |
| Gastrointestinal | 4.13E−02 | 78 |
| Endocrine System Disorders | 4.13E−02 | 55 |
| Metabolic Disease | 4.13E−02 | 63 |
| Genetic Disorder | 4.19E−02 | 127 |

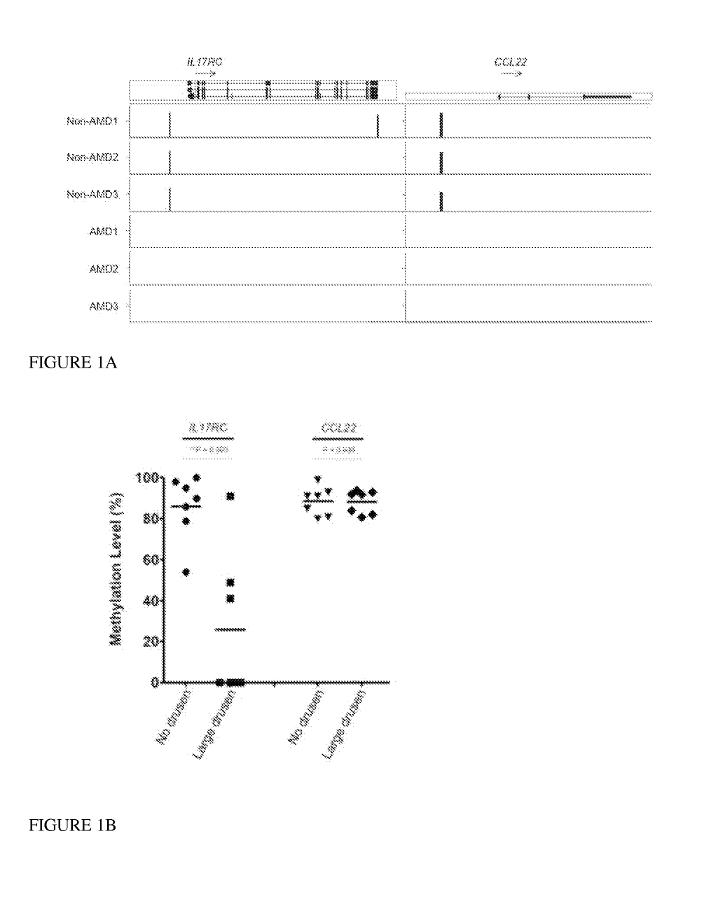

Elevated serum levels of Th17 cytokines, IL-17A and IL-22, in AMD patients (32) were demonstrated. In addition, a previous study has indicated the induction of IL-17 by complement C5a (33). Therefore, among our list of 231 genes with differential methylation patterns between twin pairs, focus was on molecules contributing to Th17 immunity. Intriguingly, as shown in FIG. 1A, MeDIP-chip data suggested that methylated CpG sites were only found in the twins without AMD but not in their AMD co-twins in the promoter regions of IL17RC, which encodes the receptor for IL-17A and IL-17F dimers, as well as CCL22, which is the chemokine mediating trafficking of activated T lymphocytes to inflammatory sites.

To test whether a similar DNA methylation pattern could be found in other AMD patients with more diverse genetic backgrounds, 7 pairs of siblings were identified who had discordant AMD phenotypes (for all sibling pairs, one of the two had large drusen greater than 125 micron in diameter in at least one eye, and the other had no drusen). Methyl-Profiler assays were carried out to detect the methylation status on the promoters of IL17RC and CCL22 (FIG. 1B). Intriguingly, a significant differential methylation status between siblings with discordant AMD was found on the promoter of IL17RC (P=0.003, Mann-Witney test). A hypomethylated IL17RC promoter was only found in the DNA from the siblings with AMD similar to that observed in the discordant twins.

Hypo-methylated Promoter if the IL17RC gene in AMD Patients

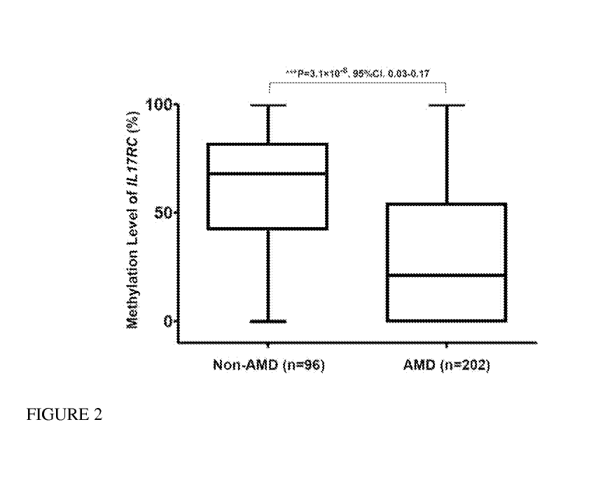

To investigate the methylation status of IL17RC promoters in patients and healthy controls who do not share known genetic similarities, genomic DNA was collected from the PBMCs of 202 AMD patients (95 wet-AMD and 107 dry-AMD) as well as 96 healthy individuals without AMD (non-AMD). The average age of AMD patients and non-AMD controls were 80.5 and 70.2 years old, respectively (Table 1, Case-Control Patient Cohort). Similar to the findings with both twins and siblings, a hypomethylated IL17RC promoter was also found in AMD patients, as compared to a hypermethylated IL17RC promoter in non-AMD controls (95% CI, 0.03-0.17, P=3.1×10⁻⁸, multivariable logistic regression adjusted for age and gender) (FIG. 2). Collectively, general association was shown of a hypomethylated IL17RC promoter with AMD.

Elevated Expression of IL-17RC in AMD Patients' Peripheral Blood

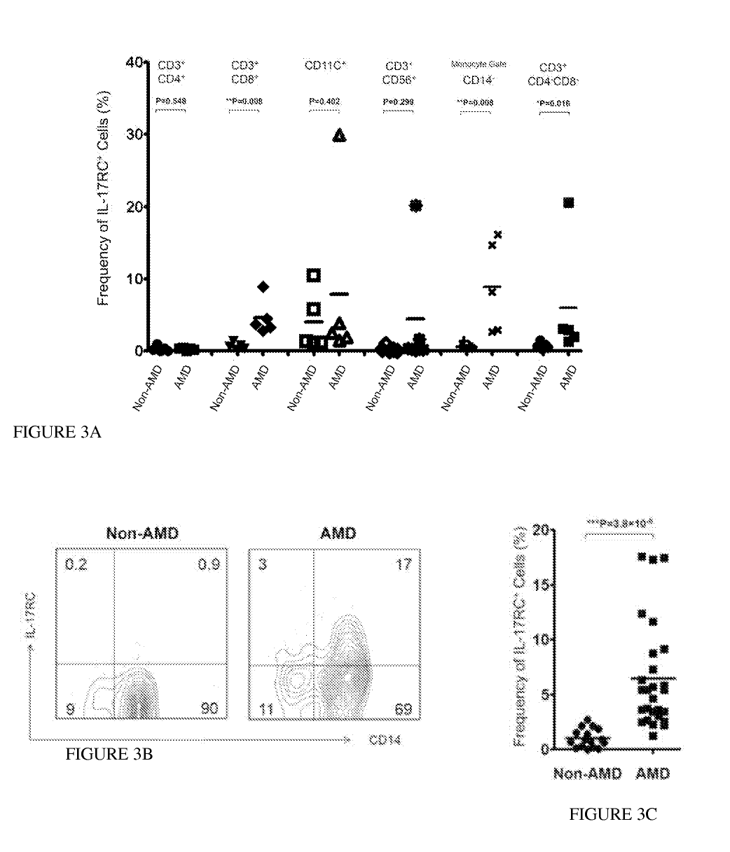

DNA methylation generally associates with gene silencing. To test whether DNA hypomethylation found on the promoter of IL17RC resulted in gene de-silencing and hence increased expression in only AMD patients, FACS analysis of whole blood samples was performed, together with other cell surface markers identifying peripheral hematopoietic cell lineages. Increased levels of IL-17RC expression were found on several types of cells from AMD patients as compared to age matched non-AMD controls, including CD3⁺CD4⁻CD8⁻ and CD8⁺ T cells, but not on CD4⁺ T cells (FIG. 3A). Intriguingly, a significantly elevated frequency of IL-17RC⁺CD14⁺ monocytes in the peripheral blood of both dry and wet AMD patients was found (FIGS. 3B and 3C). These data suggest that the hypomethylation of IL17RC promoter results in the elevated expression of the IL-17RC protein on selected cells in peripheral blood of AMD patients.

Elevated Expression of IL17RC in AMD Patients' Eyes

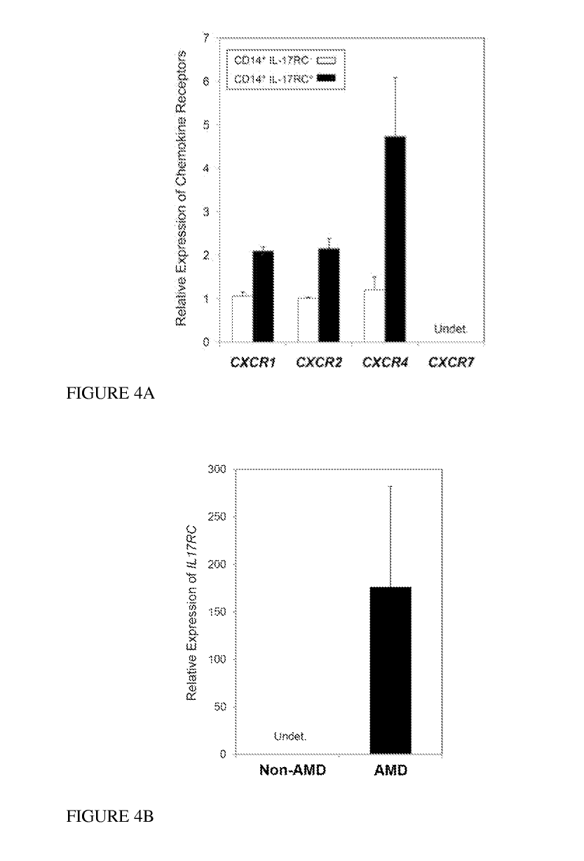

To investigate the characteristics of IL-17RC⁺CD14⁺ monocytes found only in AMD patients, the expression of CXCR4 and CXCR7, which encode the receptors for CXCL12, as well as the expression of CXCR1 and CXCR2, which encode the receptors for IL-8, in the sorted IL-17RC⁺CD14+ monocytes comparing to the sorted IL-17RC⁻CD14⁺ monocytes. As shown in FIG. 4A, the expression of CXCR1 and CXCR2 in IL-17RC⁺CD14⁺ monocytes were increased by 2 fold as compared to IL-17RC⁺CD14⁻ monocytes, while the expression of CXCR4 in IL-17RC+CD14+ monocytes was over 4 fold higher than its expression in IL-17RC⁻CD14⁺ monocytes. The expression of CXCR7 was undetectable in either of these cells. These data suggest that IL-17RC⁺CD14⁺ monocytes may have a stronger potential to migrate into the eye than the IL-17RC⁺CD14⁻ monocytes.

To test whether IL17RC expression could be found in the retinal and choroidal tissues of AMD patients, formalin-fixed, paraffin-embedded (FFPE) archived retina slides were obtained from two patients with the wet form of AMD as well as two age-matched control eyes. Quantitative real-time PCR was performed to detect the expression of IL17RC mRNA. Interestingly, IL17RC was only detectable in the retina and choroid of AMD patients but not the non-AMD controls (FIG. 4B). Thus elevated IL17RC expression was found not only in the peripheral blood but also in the eyes of AMD patients.

Twin and sibling designs are broadly used in studies dissecting the genetic versus environmental contributions to various diseases. Early studies using small patient cohorts indicated a significantly higher concordance rate of AMD in monozygotic than in dizygotic twins or families and strongly suggest a genetic predisposition to AMD. See Meyers S M. A twin study on age-related macular degeneration. Trans Am Ophthalmol Soc 1994; 92:775-843; Gottfredsdottir M S, Sverrisson T, Musch D C, Stefansson E. Age related macular degeneration in monozygotic twins and their spouses in Iceland. Acta Ophthalmol Scand 1999; 77:422-5; Klein M L, Mauldin W M, Stoumbos V D. Heredity and age-related macular degeneration. Observations in monozygotic twins. Arch Ophthalmol 1994; 112:932-7; Heiba I M, Elston R C, Klein B E, Klein R. Sibling correlations and segregation analysis of age-related maculopathy: the Beaver Dam Eye Study. Genet Epidemiol 1994; 11:51-67. In two recent twin studies, Hammond et al. showed that the concordance for AMD in monozygotic twins was 0.37 compared with 0.19 in dizygotic twins, while Seddon et al. suggested that genetic factors can explain 46% to 71% of the variation in the overall severity of AMD. Hammond C J, Webster A R, Snieder H, Bird A C, Gilbert C E, Spector T D. Genetic influence on early age-related maculopathy: a twin study. Ophthalmology 2002; 109:730-6; Seddon J M, Cote J, Page W F, Aggen S H, Neale M C. The US twin study of age-related macular degeneration: relative roles of genetic and environmental influences. Arch Ophthalmol 2005; 123:321-7. However, the later study also elucidated that for specific macular drusen and retinal pigment epithelial characteristics, both significant genetic (0.26-0.71) and unique environmental (0.28-0.64) proportions of variance were detected. The three pairs of twins (one monozygotic and two dizygotic) in the present studies display a significant difference in clinical manifestation, providing a unique chance to study the epigenetic difference between them. The present MeDIP-chip analysis identified difference in ˜1.5% of the total CpG sites within 231 gene promoters between AMD twins (data not shown). The results also suggest that epigenetic modifications of DNA may be crucial for understanding the molecular basis of complex diseases like AMD.

AMD has been traditionally considered as a neurodegenerative disease. Recent discovery of association between AMD and SNPs in genes involving in immune responses such as CFH, CFB, CFI, C3, ARMS2/HTRA1, TIMP3, TLR3, and TLR4 argues that disregulated immune system may play an important role during onset and sustain of AMD pathology. Swaroop A, Chew E Y, Rickman C B, Abecasis G R. Unraveling a multifactorial late-onset disease: from genetic susceptibility to disease mechanisms for age-related macular degeneration. Annu Rev Genomics Hum Genet 2009; 10:19-43. Interestingly, the list of genes with differential methylation patterns on their promoters between discordant AMD twins (data not shown), identified by the present MeDIP-chip analysis, also suggested potential involvement of immune system in AMD etiology.

IL-17RC protein is found highly expressed on endothelium and epithelium of lung, intestine, kidney, prostate, and joint. However, it is not clear what types of cells express IL-17RC in human peripheral blood or eyes. IL-17RC protein serves as an essential subunit of IL-17 receptor complex that mediates the signal transduction and inflammatory activities of IL-17A and IL-17F. Its binding affinity and ligand preference, as well as tissue specific distribution are slightly different between the murine and human systems. Recent studies have suggested that high levels of IL-17RC protein expression are found in prostate cancer cells, as well as synoviocytes and blood cells from patients with rheumatoid arthritis. In the present studies, hypomethylated IL17RC promoter was found in patients with both wet and dry AMD as compared to non-AMD controls, which resulted in the increased expression of its protein in CD14⁺ monocytes. Moreover, it was demonstrated that IL-17RC⁺CD14⁺ monocytes expressed elevated CXCR4, CXCR1 and CXCR2, which may promote their trafficking into the inflammatory sites within retinal and choroidal tissues in AMD patients and eventually causing the pathology of AMD. Therefore, these results suggest a potential mechanism by which proinflammatory monocytes could promote AMD pathology.

It has heretofore been identified that DNA hypo-methylation of promoter of the IL17RC gene in AMD patients leads to elevated expression of its protein and mRNA in peripheral blood and retina tissues. Thus, promoter DNA methylation pattern and expression of IL-17RC protein can serve as a biomarker for AMD diagnosis. Currently, anti-Vascular Endothelial Growth Factor (anti-VEGF) drugs is the only available drug for the treatment of neovascular wet AMD.

The present studies suggest that soluble IL-17RC molecules, which are known in the art, for example, such as those soluble IL-17RC molecules described in U.S. Published Application No. 2008/0305078, which could act as decoy receptors to dampen signalling of IL-17 cytokines, can be a new therapy for AMD. Thus, one embodiment of the invention is directed to methods of treating age-related macular degeneration in a patient comprising administering to the patient a therapeutically effective amount of a soluble IL-17RC molecule.

As used herein, a “therapeutically effective amount” is an amount effective, at dosages, and for periods of time necessary, to achieve the desired result with respect to the treatment of the relevant disorder. It will be appreciated that the effective amount of soluble IL-17RC will vary from patient to patient not only with the particular compound, component or composition selected, the route of administration, and the ability of the components to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. As an example, the soluble IL-17RC molecules useful in the methods of the present invention are administered at a dosage and for a time such that the level of activation and adhesion activity of platelets is reduced as compared to the level of activity before the start of treatment.

The soluble IL-17RC molecules of the invention may be administered in an effective amount by any of the conventional techniques well-established in the medical field. The soluble IL-17RC molecules employed in the methods of the present invention including, for example, the soluble IL-17RC molecules of the invention may be administered by any means that results in the contact of the active agents with the agents' site or sites of action in the body of a patient. The soluble IL-17RC molecules may be administered by any conventional means available.

### EXPERIMENTAL SECTION

### Methods

Patients

All protocols were approved by institutional review boards, and written informed consent for epigenetic and genetic testing and participation was provided by the patients to the National Institutes of Health (NIH), Casey Eye Institute (CEI) (Oregon Health and Science University), or Centre for Eye Research Australia (CERA) through the Royal Victorian Eye and Ear Hospital (RVEEH) Human Research and Ethics Committee and the Australian Twin Registry (ATR) approved the project. Twin volunteers, both identical and non-identical with or without known eye disease, of either gender, aged 18 years or older were invited to participate through the ATR. The non-AMD controls were from the NIH eye clinics and were screened for AMD. All patients and non-AMD controls are Caucasian. The age and gender information was listed in Table 1.

DNA Methylation Microarray (MEDIP-chip) and Data Analysis

DNA sample preparation and hybridization to DNA methylation microarray were performed according to the manufacture's protocols provided by Roche-NimbleGen, WI. Briefly, Genomic DNA is isolated from peripheral blood mononuclear cells (PBMCs) using the Qiagen DNeasy kit (Qiagen, CA). Six μg total DNA was then digested with Msel endonuclease (NEB, MA) overnight to produce small fragments (200-1000 bp). Antibody against 5-methyl cytidine (Ab10805, Abcam, MA) was used to immunoprecipitate methylated DNA fragments. Enriched methylated DNA and input DNA was amplified using WGA2 GenomePlex Complete Whole Genome Amplification Kit (Sigma, Mo.) according to manufacture's protocol and sent to Roche-NimbleGen for labelling and hybridization to Human DNA Methylation 2.1M Deluxe Promoter Arrays. Data analysis service was provided by NimbleGen. All raw data were deposited in GEO with accession number GSE28033.

Detection of DNA Methylation on Selected Gene Promoters

One μg total genomic DNA from PBMCs was digested for 6 hours at 37° C. using the Methyl-Profiler DNA Methylation Enzyme Kit (SABiosciences, MD) according to the manufacture's instruction. Quantitative Real-time PCR was performed with an ABI PRISM 7900HT Sequence Detection System (Applied Biosystems, CA). The percentage of methylated DNA was calculated by the difference of CT cycle numbers between samples with total undigested DNA and methylation-sensitive restriction enzyme digested DNA. The following primers were used:

CCL22F: ACCACCACACCCAGCTAAAT,
CCL22R: CACGCCCTGCT-CATTTTTAT;
IL17RCF: CCAAGATCCGGAAAGATGAA,
IL17RCR: GCATCT-TCTAGGTGCCAGGT.

Detection of Cell Surface Molecules by Flow Cytometry

Whole blood, collected in Na-heparin vacutainers (BD, CA), from AMD patients or healthy volunteers were first subjected to Red Blood Cell lysis using ACK Lysis buffer (Quality Biological Inc, MD). Cells were then incubated in sterile PBS supplemented with 10% FCS and fluorochrome-conjugated antibodies against CD3, CD4, CD8, CD14, CD45, CD11C, CD56 (all from BD, CA) as well as IL-17RC (FAB22691A, R&D, MN), for 30 minutes at room temperature in dark. Cells were washed three times and analyzed on LSR-II analyzer (BD, CA). All flow cytometry data were analysed using FlowJo 9.1 (Treestar, OR).

Detection of IL17RCExpression in Human Retinal and Choroidal Tissues

Archived formalin-fixed paraffin-embedded (FFPE) samples were first washed in 100% Xylene for 10 times, followed by sequential washes in 100% ethanol, 95% ethanol, 75% ethanol, and water (10 times in each solution). Total RNA was isolated from retina tissues of two AMD patients as well as two age-matched controls, using mirVana miRNA kit (Ambion, Tex.). Reverse transcription of RNA was performed for 12 hours at 37° C., followed by Quantitative Real-time PCR, using the primers and probes for 18s rRNA (Hs03928990_g1) and IL17RC (Hs00994305_m1) from Applied Biosystems. The comparative threshold cycle method and 18s rRNA was used to normalize the expression of the IL17RC.

Genetic Analysis

Genomic DNA was extracted from PBMCs of each individual using the Genomic DNA Purification kit (Promega, WI). DNA samples were analyzed by TaqMan genotyping assays using the ABI PRISM 7500 sequence detection system (Applied Biosystems, CA). The primers and probes for ARMS2 rs10490924 was from the inventory SNP assay while CFHrs1061170 and HTRA1 rs11200638 were custom-designed from Applied Biosystems. Genotypes were determined based on the fluorescence intensities of FAM and VIC. The call rates of 3 assays were >98.5% and the call accuracies (consistency of duplicate wells of selected samples) were 100%.

Statistical Analysis

Statistical analysis was performed using GraphPad Prism 5.0 software (GraphPad, La Jolla, Calif.). The Mann-Whitney test was used to compare differences between two groups and the significance level was set at P<0.05. Multivariable logistic regression analysis was used to detect the difference of the IL17RC promoter methylation status, adjusted by age, gender, genotypes of CFH, ARMS2, or HTRA1, using SAS 9.2. The significance level was set at P<0.05.

### REFERENCES

1. Friedman DS, O'Colmain B J, Munoz B, et al. Prevalence of age-related macular degeneration in the United States. Arch Ophthalmol 2004; 122:564-72.
2. Swaroop A, Chew E Y, Rickman C B, Abecasis G R. Unraveling a multifactorial late-onset disease: from genetic susceptibility to disease mechanisms for age-related macular degeneration. Annu Rev Genomics Hum Genet 2009; 10:19-43.
3. Edwards A O, Ritter R, 3rd, Abel K J, Manning A, Panhuysen C, Farrer L A. Complement factor H polymorphism and age-related macular degeneration. Science 2005; 308:421-4.
4. Haines J L, Hauser M A, Schmidt S, et al. Complement factor H variant increases the risk of age-related macular degeneration. Science 2005; 308:419-21.
5. Klein R J, Zeiss C, Chew E Y, et al. Complement factor H polymorphism in age-related macular degeneration. Science 2005; 308:385-9.
6. Hageman G S, Anderson D H, Johnson L V, et al. A common haplotype in the complement regulatory gene factor H (HF1/CFH) predisposes individuals to age-related macular degeneration. Proc Natl Acad Sci USA 2005; 102:7227-32.
7. Maller J, George S, Purcell S, et al. Common variation in three genes, including a noncoding variant in CFH, strongly influences risk of age-related macular degeneration. Nat Genet 2006; 38:1055-9.
8. Gold B, Merriam J E, Zernant J, et al. Variation in factor B (BF) and complement component 2 (C2) genes is associated with age-related macular degeneration. Nat Genet 2006; 38:458-62.
9. Yates J R, Sepp T, Matharu B K, et al. Complement C3 variant and the risk of age-related macular degeneration. N Engl J Med 2007; 357:553-61.
10. Fagerness J A, Maller J B, Neale B M, Reynolds R C, Daly M J, Seddon J M. Variation near complement factor I is associated with risk of advanced AMD. Eur J Hum Genet 2009; 17:100-4.
11. Dewan A, Liu M, Hartman S, et al. HTRA1 promoter polymorphism in wet age-related macular degeneration. Science 2006; 314:989-92.
12. Yang Z, Camp N J, Sun H, et al. A variant of the HTRA1 gene increases susceptibility to age-related macular degeneration. Science 2006; 314:992-3.
13. Fritsche L G, Loenhardt T, Janssen A, et al. Age-related macular degeneration is associated with an unstable ARMS2 (LOC387715) mRNA. Nat Genet 2008; 40:892-6.
14. Kanda A, Chen W, Othman M, et al. A variant of mitochondrial protein LOC387715/ARMS2, not HTRA1, is strongly associated with age-related macular degeneration. Proc Natl Acad Sci USA 2007; 104:16227-32.
15. Baird P N, Richardson A J, Robman L D, et al. Apolipoprotein (APOE) gene is associated with progression of age-related macular degeneration (AMD). Hum Mutat 2006; 27:337-42.
16. Chen W, Stambolian D, Edwards A O, et al. Genetic variants near TIMP3 and high-density lipoprotein-associated loci influence susceptibility to age-related macular degeneration. Proc Natl Acad Sci USA 2010; 107:7401-6.
17. Zareparsi S, Buraczynska M, Branham K E, et al. Toll-like receptor 4 variant D299G is associated with susceptibility to age-related macular degeneration. Hum Mol Genet 2005; 14:1449-55.
18. Yang Z, Stratton C, Francis P J, et al. Toll-like receptor 3 and geographic atrophy in age-related macular degeneration. N Engl J Med 2008; 359:1456-63.
19. Neale B M, Fagerness J, Reynolds R, et al. Genome-wide association study of advanced age-related macular degeneration identifies a role of the hepatic lipase gene (LIPC). Proc Natl Acad Sci USA 2010; 107:7395-400.
20. Peter I, Seddon J M. Genetic epidemiology: successes and challenges of genome-wide association studies using the example of age-related macular degeneration. Am J Ophthalmol 2010; 150:450-2 e2.
21. de Jong P T. Age-related macular degeneration. N Engl J Med 2006; 355:1474-85.
22. Javierre B M, Fernandez A F, Richter J, et al. Changes in the pattern of DNA methylation associate with twin discordance in systemic lupus erythematosus. Genome Res 2009; 20:170-9.
23. Baranzini S E, Mudge J, van Velkinburgh J C, et al. Genome, epigenome and RNA sequences of monozygotic twins discordant for multiple sclerosis. Nature 2010; 464:1351-6.
24. Kaminsky Z A, Tang T, Wang S C, et al. DNA methylation profiles in monozygotic and dizygotic twins. Nat Genet 2009; 41:240-5.
25. Fraga M F, Ballestar E, Paz M F, et al. Epigenetic differences arise during the lifetime of monozygotic twins. Proc Natl Acad Sci USA 2005; 102:10604-9.
26. Meyers S M. A twin study on age-related macular degeneration. Trans Am Ophthalmol Soc 1994; 92:775-843.
27. Gottfredsdottir M S, Sverrisson T, Musch D C, Stefansson E. Age related macular degeneration in monozygotic twins and their spouses in Iceland. Acta Ophthalmol Scand 1999; 77:422-5.
28. Klein M L, Mauldin W M, Stoumbos V D. Heredity and age-related macular degeneration. Observations in monozygotic twins. Arch Ophthalmol 1994; 112:932-7.
29. Heiba 1M, Elston R C, Klein B E, Klein R. Sibling correlations and segregation analysis of age-related maculopathy: the Beaver Dam Eye Study. Genet Epidemiol 1994; 11:51-67.
30. Hammond C J, Webster A R, Snieder H, Bird A C, Gilbert C E, Spector T D. Genetic influence on early age-related maculopathy: a twin study. Ophthalmology 2002; 109:730-6.
31. Seddon J M, Cote J, Page W F, Aggen S H, Neale M C. The US twin study of age-related macular degeneration: relative roles of genetic and environmental influences. Arch Ophthalmol 2005; 123:321-7.
32. Chang S H, Dong C. Signaling of interleukin-17 family cytokines in immunity and inflammation. Cell Signal; 2010:2.
33. Ho A W, Gaffen S L. IL-17RC: a partner in IL-17 signaling and beyond. Semin Immunopathol 2009; 32:33-42.
34. Ge D, You Z. Expression of interleukin-17RC protein in normal human tissues. Int Arch Med 2008; 1:19.
35. Campa C, Harding S P. Anti-VEGF Compounds in the Treatment of Neovascular Age Related Macular Degeneration. Curr Drug Targets 2010; 2010:1.
36. Group TA-REDSR. A randomized, placebo-controlled, clinical trial of high-dose supplementation with vitamins C and E, beta carotene, and zinc for age-related macular degeneration and vision loss: AREDS report no. 8. Arch Ophthalmol 2001; 119:1417-36.
37. Krishnadev N, Meleth A D, Chew E Y. Nutritional supplements for age-related macular degeneration. Curr Opin Ophthalmol 2010; 21:184-9.

What is claimed:

1. A method of diagnosing the presence, severity, or predisposition for development of age-related macular degeneration in a patient comprising:
determining the methylation of the promoter of IL17RC gene in the DNA from a patient having, suspected of having, or suspected to be at risk for age-related macular degeneration;
determining whether the promoter of IL17RC gene is less methylated relative to the methylation of the promoter of IL17RC gene in a control from an individual or individuals not having age-related macular degeneration, the less methylation being indicative of the presence, severity, or predisposition for development of age-related macular degeneration in the patient.

2. A method of diagnosing the presence, severity, or predisposition for development of age-related macular degeneration in a patient comprising:
determining the frequency of IL-17RC⁺ cells in a whole blood sample from a patient having, suspected of having, or suspected to be at risk for age-related macular degeneration;
determining whether the blood sample has an elevation in the frequency of IL-17RC⁺ cells relative to the frequency of IL-17RC⁺ cells in a control from an individual or individuals not having age-related macular degeneration, the elevation being indicative of the presence, severity, or predisposition for development of age-related macular degeneration in the patient.

3. A method of treating age-related macular degeneration in a patient comprising administering to the patient a therapeutically effective amount of a soluble IL-17RC molecule.
